(19) United States
(12) Patent Application Publication
(10) Pub. No.: US 2022/0280112 A1
(43) Pub. Date: Sep. 8, 2022

(54) METHOD FOR DETERMINATION OF SENSOR LOCALIZATION ON THE BODY OF A USER

(71) Applicant: PD Neurotechnology LTD, London SE (GB)

(72) Inventor: Dimitrios FOTIADIS, Athens (GR)

(21) Appl. No.: 17/776,830

(22) PCT Filed: Nov. 14, 2019
(86) PCT No.: PCT/GR2019/000079

§ 371 Date: May 13, 2022

(51) Int. Cl. A61B 5/00; A61B 5/11

(52) U.S. Cl. CPC A61B 5/684; A61B 5/6823; A61B 5/6824; A61B 5/6829; A61B 5/002; A61B 5/1118; A61B 2562/0219

(57) ABSTRACT

Method and system for determining the localization of wearable sensors on the body of a user among a number of predefined attachment sites, comprising collecting kinematic data from at least two Inertial Measurement Units (IMUs) embedded in wearable devices attached to a user, transferring all the signals collected to a separate processing unit, comprising a memory and a comparator engine, and comparing signal characteristics to determine the sites of attachment to the user. Said system and method are useful for the monitoring of movement disorders such as Parkinson's disease.

### TECHNICAL FIELD

The present invention relates to a method and system for determination of the localization of wearable sensors on the body of a user.

### BACKGROUND OF THE INVENTION

Body sensor networks (BSN) have been widely used in literature for monitoring and studying movement disorders and rehabilitation. Those devices typically include Inertial Measurement Units (IMU) with one or more accelerometers, gyroscopes and magnetometers among other sensors. IMUs are used to quantify motion and extract motion features that are correlated with movement disorders measured in specific controlled tasks or during daily activities (walking, dressing etc.). However, the majority of commercial systems are research oriented and designed to be operated by technicians or researchers. The correct placement of the sensors on the designated body parts and the recording are performed by technicians who keep track of the placement of each sensor and label them accordingly to ensure the proper processing of the signals collected. In some cases where a configuration is not required prior to mounting the devices on the body, there is a calibration phase where the user, after having the devices mounted, must perform specific activities/tasks for each device to identify its position.

However, neither labels nor calibration tasks are practical or even feasible when BSNs are intended for home use and users who may be of old age or suffer from cognitive impairments.

Therefore, the present invention describes a method for automatic identification of sensor position when the devices embedding them are mounted on a group of predefined body parts using kinematic data from daily activities of the user. It nullifies the need for configuring the devices prior to mounting them or performing specific calibration tasks after mounting them on the user's body. This invention could make devices based on BSNs more user friendly without requiring extra steps for application.

### SUMMARY OF THE INVENTION

The invention described herein provides a method and system that enables the determination of the sites of attachment of wearable sensors on the body of a user, wherein the sites of attachment are selected among a number of predefined attachment sites. More specifically, the proposed system and method collects kinematic data from at least two Inertial Measurement Units (IMUs) embedded in separate wearable devices attached to a user, transfers all the signals collected to a separate processing unit, comprising a memory and a comparator engine, and compares signal characteristics to determine the sites of attachment to the user, wherein said sites are selected among a predefined group of body parts.

Using IMU sensors to extract accurate kinematic features, such as gait parameters including but not limited to swing and stance phase, toe-off and heel-off events, stride length and duration, double limb support and single limb support, Parkinson's disease related symptoms including but not limited to tremor, bradykinesia and dyskinesia severity, freezing of gait, and activity states including but not limited to walking, lying, standing and sitting periods, heavily depends on the identification of the positioning of their placement on the subject's body. Regarding body sensor networks in particular, where multiple sensors are attached to various body parts, it is essential to know the placement of each sensor to construct a biomechanical model as precisely as possible, and consider different body parts' range and angles of motion. However, keeping the acquisition mode of all sensors the same for all attached devices, i.e., not changing it depending on the particular site of attachment, allows for processing the most detailed, unfiltered, raw signal after the collection. Thus, the system and method for identification of sensor body positioning proposed herein is suitable for medical and research applications, and particularly for the monitoring of movement disorders, where accuracy is very important in order to identify abnormalities and irregular patterns of movement.

Another potential advantage of the system and method provided in the present invention is that it simplifies the use of wearable devices, because the determination of sensor localization is achieved in an automated rather than a manual fashion. Thus, the subject in not required to perform the task of specifying which sensor is attached to which part of the body, a task which is particularly complicated when multiple wearable devices are being used.

An additional and related advantage of the present system and method is that they potentially reduce user error, as they minimize the amount of input needed from the subject. This is especially important when the wearable devices are being used by certain patient groups such as the elderly or people with reduced mental capacity.

Other aspects and benefits of the present invention will become apparent from the detailed description to follow.

### BRIEF DESCRIPTION OF THE DRAWINGS

The present invention will now be described with reference to certain embodiments thereof which are illustrated in the accompanying drawings. It should be noted that the accompanying drawings illustrate preferred embodiments of the invention, therefore should not be considered as limiting the scope of the invention.

FIG. 1 is a schematic presentation of the steps taken for the determination of sensor position for 2 sensors

FIG. 2 is a schematic presentation of the steps taken for the determination of sensor position for 3 sensors

FIG. 3 is a schematic presentation of the steps taken for the determination of sensor position for 4 sensors

FIG. 4 is a schematic presentation of the steps taken for the determination of sensor position for 5 sensors

FIG. 5 is a schematic presentation of the orientation of the axes considered in the proposed invention, considering the subject in standing position and the body part(s) where the device(s) is/are mounted looking downwards.

### DETAILED DESCRIPTION OF THE INVENTION

The present disclosure provides a system and method for collecting kinematic data and monitoring kinematic features comprising: a) collecting kinematic data from at least two Inertial Measurement Units (IMUs) embedded in wearable devices attached to a user, b) transferring all the signals collected to a separate processing unit, comprising a memory and a comparator engine and c) comparing signal characteristics to determine the specific sites of attachment to the user, wherein said sites are selected among a predefined group of body parts.

The term “kinematic data” as used throughout the description and claims refers to the signals collected using a wearable IMU sensor, comprising one or more accelerometers, gyroscopes and magnetometers. The kinematic data include but are not limited to acceleration, rotation rate and magnetic flux.

The term “kinematic features” as used throughout the description and claims refers to human movement patterns and events, such as gait parameters including but not limited to swing and stance phase, toe-off and heel-off events, stride length and duration, double limb support and single limb support, Parkinson's disease related symptoms including but not limited to tremor, bradykinesia and dyskinesia severity, freezing of gait, and activity states including but not limited to walking, lying, standing and sitting periods.

The inventors have unexpectedly found that by letting all sensors collect signal in their full capacity and not defining different monitoring modes or activity states of interest during collection, the resulting signals are not filtered or processed in any way prior to processing them all together after the collection sessions. This provides with unfiltered, full resolution, information-rich signals, which could be valuable kinematic data that could be attenuated and ultimately lost by any kind of processing during collection.

Suitable IMUs comprise one or more accelerometers, gyroscopes, and magnetometers, among others. Preferably, the IMUs comprise a 3-axis accelerometer and a 3-axis gyroscope.

The comparator engine may be part of a docking station where wearable devices are connected either with a physical/cable connection (i.e. USB or custom connectors) or wirelessly (i.e. Bluetooth, Zigbee) and is used both for data processing and charging of the wearable devices. The docking station may have a processing unit, memory and internal storage for running the comparator engine and processing and storage of the data acquired by the wearable devices. The docking station may also have a WiFi and/or Ethernet connection for uploading raw and/or processed data to a cloud application or a dedicated server. Alternatively, the separate processing unit with the memory and comparator engine required by the method proposed may be a mobile device, phone or tablet, with a dedicated application installed for processing and transferring the data.

The characterization and determination of sensor localization according to the body part they are attached to occurs during the post-processing of the signals collected.

Post-processing occurs when recording is finished which is marked either by putting devices on a docking station, by stopping recording with a specific purpose software either as mobile or desktop application, or by pressing a button on one or more devices.

According to the present invention, a full resolution raw IMU signal is collected from all wearable devices; processing by the comparator is performed only post-collection. That allows for the method or system to be used in applications where very high accuracy and no signal loss is a requirement, such as but not limited to health applications. The characterization of each sensor as worn on a specific body part is done post-processing, which means that the sensor during collection is not optimized for a specific body part, because the steps of applying filtering, averaging, windowing or any other processing while collecting could ultimately attenuate signal characteristics that indicate impaired movement and smother pathological patterns in kinematic data, which could have only slight variations from healthy ones, but which are very valuable for a biomedical application. Dealing with the position identification only after signal collection, during post-processing, ensures that the signal collected has full resolution and is as detailed and raw as possible, allowing the application of algorithms tailored to identifying kinematic features and patterns related to specific conditions, such as movement disorders.

Thus, the system and method of the present invention can be used in the monitoring of movement disorders. Said movement disorders include but are not limited to

Parkinson's disease, Huntington's disease, essential tremor, Tourette's syndrome, epilepsy, dystonia, multiple sclerosis and cerebral palsy.

IMU sensor data from all devices are initially synchronized. Time synchronization is performed offline based on each device's real time clock. Alternatively, time synchronization could be based on real time synchronization protocols with Bluetooth or Zigbee wireless communication.

A number of characteristics/features are extracted from the synchronized signals from all devices. According to one embodiment, the signal characteristics that are used by the comparator to determine the sites of attachment to the user are selected among the group comprising: the number of changes from positive to negative values along the x axis of the acceleration, the gyroscope total energy, the correlation between the x and y axes of the gyroscope and the ratio of maximum positive to maximum negative gyroscope energy on the z axis.

In an embodiment, the wearable devices are attached to predefined body parts. Preferred body parts include the torso, including the pelvic area, the chest, the clavicle area or the waist; the wrists or lower arms; and the shanks or ankles.

In a preferred embodiment, the wearable devices are attached to at least two different body parts. Preferred configurations include: one placed on the shank and one on the wrist (2 sensors); one placed on the shank, one on the torso and one on the wrist (3 sensors); two placed on the wrists and two on the shanks (4 sensors); two placed on the wrists, two on the shanks and one on the torso (5 sensors).

The comparator engine detects the total number of posture changes for all sensors, for instance the change of the accelerometer x axis from positive to negative.

After detecting the total number of posture changes for all sensors and depending on the number of sensors, the comparator identifies the positioning of each sensor, depending on the configuration of attachment sites used. The configurations discussed below are exemplary in nature and may be reconfigured without departing from the scope and spirit of the present invention.

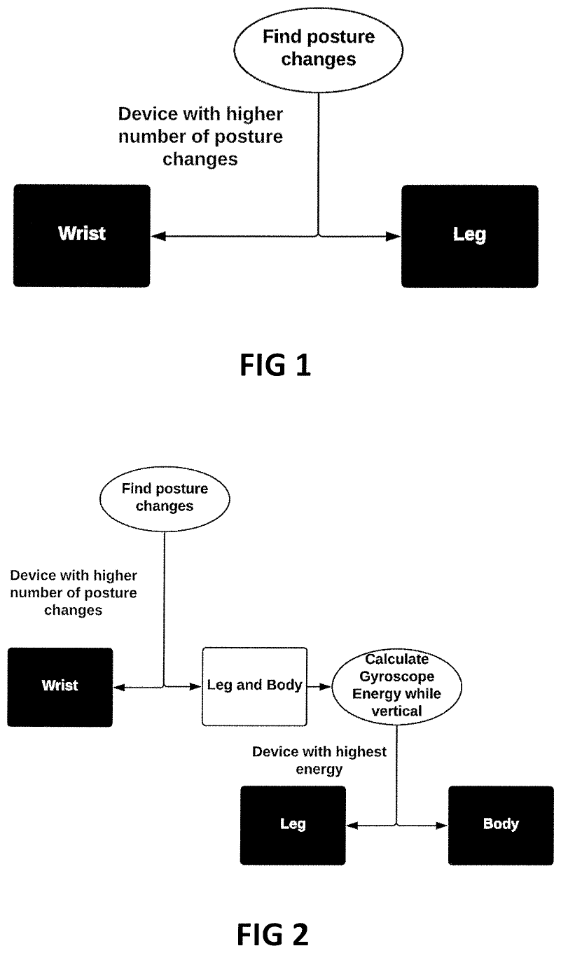

For two sensors, as in the exemplary FIG. 1 where the sensors are located one on the shank and one on the wrist, the comparator preferably uses the number of posture changes to identify the wrist sensor as the one with the most posture changes.

For three sensors, one on the shank, one on the torso and one on the wrist, as shown in FIG. 2, the comparator preferably uses the number of posture changes to identify the wrist sensor as the one with the most changes and then uses the gyroscope energy while vertical to identify the shank sensor as the one with the highest energy.

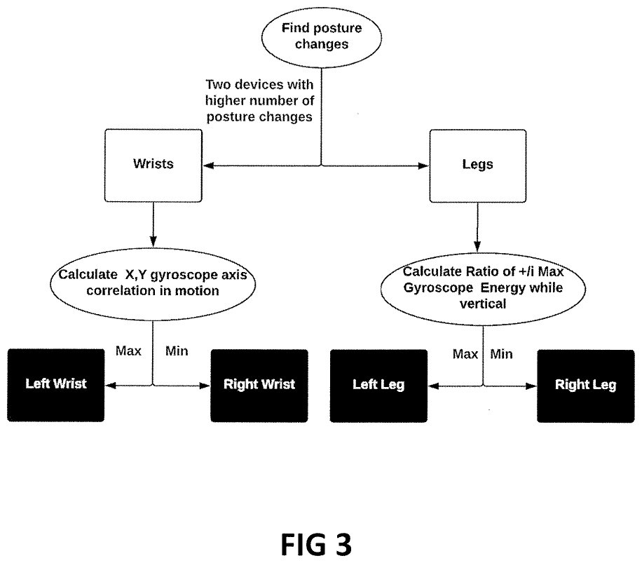

For four sensors, two on the wrists (left and right) and two on the shanks (left and right), the comparator preferably uses the number of posture changes to identify the two wrist sensors as those with the most changes and the two shank sensors as those with the least number of changes. It then calculates the correlation between the x and y gyroscope axes to identify the left wrist sensor as the one where the correlation is positive and the right wrist sensor as the one where the correlation is negative. To identify the right and left leg sensors, the comparator preferably uses the ratio of maximum positive and maximum negative gyroscope energy on the z axis, where the right shank is expected to have maximum energy on the positive part of the z axis when walking (vertical position) and the left shank is expected to have maximum energy on the negative part of the z axis. This exemplary arrangement of 4 sensors and the steps performed to determine sensor position are depicted in FIG. 3.

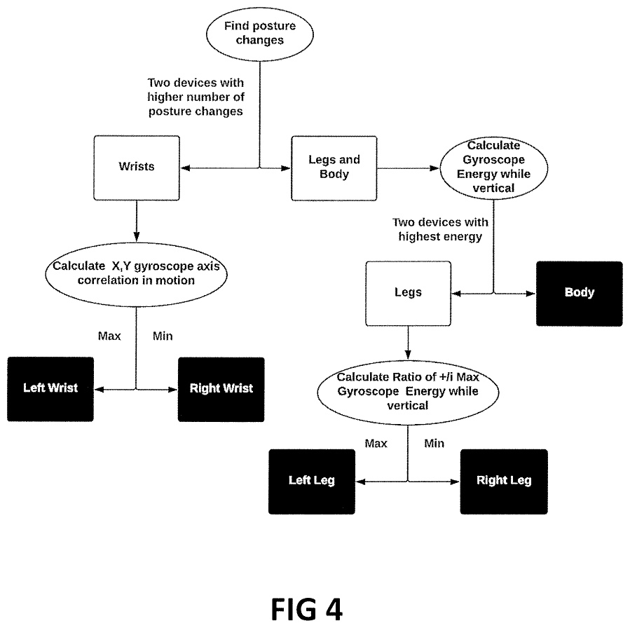

For five sensors in the configuration shown in FIG. 4, i.e. two on the wrists (left and right), two on the shanks (left and right) and one on the torso, the comparator preferably uses the number of posture changes to identify the two wrist sensors as those with the most changes and the two shank sensors and torso sensor as those with the least number of changes, and then calculates the correlation between the x and y gyroscope axes to identify the left wrist sensor as the one where the correlation is positive and the right wrist sensor as the one where the correlation is negative. To distinguish the shank sensors from the torso sensor the comparator preferably calculates the gyroscope energy while vertical to identify the torso sensor as the one with the lowest energy and the shank sensors as those with the highest energy. To identify the right and left leg sensors, the comparator preferably uses the ratio of maximum positive and maximum negative gyroscope energy on the z axis, where the right shank is expected to have maximum energy on the positive part of the z axis when walking (vertical position) and the left shank is expected to have maximum energy on the negative part of the z axis.

In a preferred embodiment, the at least two hardware wearables containing two IMUs attached to the body of the subject contain the same hardware.

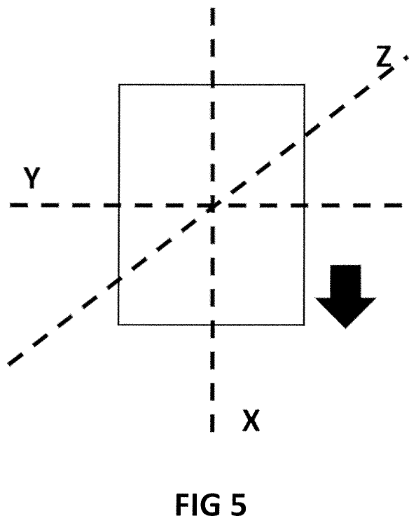

The x, y, z axes of the sensors referred to in the proposed method are always defined as shown in FIG. 5. Regardless of the actual orientation of the wearable sensor, the axes should be adapted after signal collection to match the specific orientation with the x axis pointing to the ground.

Preferably, signal collection is performed with the same sampling frequency, set as high as possible and preferably above 50 Hz. Changing the collection mode (frequency) of the sensors depending on the body part during signal collection could cause loss of information that could be relevant when monitoring movement disorders patients.

The kinematic data referred to herein are collected while the user is performing unconstrained daily activities. The subject does not need to perform specific tasks or take postures for the comparator to properly identify the sensor positioning. This is achieved using aggregated characteristics of the signals collected during the entire signal collection session, such as the number of changes from positive to negative values along the x axis of the acceleration, the gyroscope total energy, the correlation between the x and y axes of the gyroscope and the ratio of maximum positive to maximum negative gyroscope energy on the z axis.

The kinematic features that are monitored using the system and method disclosed herein comprise the full gait cycle and events, such as swing and stance phase, toe-off and heel-off events, stride length and duration, double limb support and single limb support. Parkinson's disease related symptoms are monitored as well, such as tremor, bradykinesia and dyskinesia severity, freezing of gait, and activity states, such as walking, lying, standing and sitting periods.

The wearable devices of the present invention do not need any manual means of defining the site of localization, such as specific labels, before positioning the sensors onto the predefined body parts. This reduces the number of steps that need to be taken pre-monitoring and simplifies the use of the devices by the subject.

No calibration needs to be performed for the comparator to properly identify the sensor positioning. Typically, similar methods require a calibration phase where the user should stand in a specific posture for ten or more seconds or perform a specific activity, such as arm and leg swinging, arm extensions among other activities, in order to identify the correct position. The method uses all activities and unconstrained normal body motion performed during the day to identify the correct position of each sensor.

In addition, no configuration is needed prior to wearing the devices, such as but not limited to using a dedicated software to assign a body position for each wearable device.

1. A method for collecting kinematic data and monitoring kinematic features comprising:
collecting kinematic data from two to five Inertial Measurement Units, IMUs, embedded in wearable devices attached to a user at two to five attachment sites selected from:
the torso, including the pelvic area, the chest, the clavicle area or the waist,
the left wrist or lower arm,
the right wrist or lower arm,
the left shank or ankle,
the right shank or ankle;
transferring all the kinematic data collected to a separate processing unit, comprising a memory and a comparator engine; and the comparator engine comparing kinematic data characteristics to determine for each of the IMUs its site of attachment to the user by
using an orientation of the IMU where the x axis is looking downwards,
using a number of posture changes from positive to negative values along the x axis of the acceleration to differentiate an IMU attached to the left or right wrist or lower arm from an IMU attached to the torso or left or right shank or ankle,
using gyroscope total energy to differentiate an IMU attached to the torso from an IMU attached to the left or right shank or ankle.

2. The method of according to claim 1, further including correlation between the x and y axes of the gyroscope to differentiate an IMU attached to a left wrist or lower arm from an IMU attached to a right wrist or lower arm and the ratio of maximum positive to maximum negative gyroscope energy on the z axis to differentiate an IMU attached to a left shank or ankle from an IMU attached to a right shank or ankle.

3. The method of claim 1, wherein the wearable devices are attached to at least two different predefined attachment sites, in one of the following configurations: one shank or ankle and one wrist or lower arm (2 IMUs); one shank or ankle, one torso and one wrist or lower arm (3 IMUs); two wrists or lower arms and two shanks or ankles (4 IMUs); two wrists or lower arms, two shanks or ankles and one torso (5 IMUs).

4. The method of claim 1, wherein the at least two IMUs are the same and kinematic data collection is performed with the same sampling frequency.

5. The method of claim 1 according to any one of the previous claims, wherein the kinematic data collected by the IMUs are transferred either wirelessly, using Bluetooth or other wireless transfer protocol, or through a physical connection, such as USB, to the separate processing unit, where they are the input for the comparator.

6. The method of claim 1 according to any one of the previous claims, wherein the kinematic data are collected while the user is performing unconstrained daily activities.

7. The method of claim 1 according to any one of the previous claims, wherein no calibration needs to be performed for the comparator to properly identify the sites of IMUs' attachment to the user.

8. The method of claim 1 according to any one of the previous claims, which does not require a step of configuring the IMUs before attaching the devices to the user.

9. A method for collecting kinematic data and monitoring kinematic features comprising:
collecting kinematic data from two to five Inertial Measurement Units, IMUs, embedded in wearable devices attached to a user on two to five different body parts;
the body parts being among a predefined group comprising: the torso, including the pelvic area, the chest, the clavicle area or the waist; the wrists or lower arm; and the shanks or ankles;
the devices being attached to two to five different predefined body parts, in one of the following configurations:
one shank or ankle and one wrist or lower arm (2 sensors);
one shank or ankle, one torso, including the pelvic area, the chest, the clavicle area or the waist, and one wrist or lower arm (3 sensors);
two wrists or lower arms and two shanks or ankles (4 sensors);
two wrists or lower arms, two shanks or ankles and one torso , including the pelvic area, the chest, the clavicle area or the waist (5 sensors);
collecting kinematic data while the user is performing unconstrained daily activities;
transferring all the kinematic data collected to a separate processing unit, comprising a memory and a comparator engine;
extracting signal characteristics from the kinematic data selected among the group comprising: the number of changes from positive to negative values for all the axes of the acceleration, the gyroscope total energy, the correlation between the axes of the gyroscope and the ratio of maximum positive to maximum negative gyroscope energy for all axes; and
comparing signal characteristics to determine the body parts of attachment to the user.

10. The method of claim 9, wherein all the IMUs used in the devices are the same in terms of technical specifications and performance characteristics, and kinematic data collection is performed with the same sampling frequency.

11. The method of claim 9, wherein the kinematic data collected by the sensors are transferred either wirelessly, using Bluetooth or other wireless transfer protocol, or through a physical connection, such as USB, to the processing unit, where they are the input for the comparator.

12. The method of claim 9, wherein said kinematic features comprise gait parameters, including but not limited to swing and stance phase, toe-off and heel-off events, stride length and duration, double limb support and single limb support; Parkinson's disease related symptoms, including tremor, bradykinesia and dyskinesia severity, freezing of gait; and activity states, including walking, lying, standing and sitting periods.

13. The method of claim 9, comprising with the processing unit, determine for each of the IMUs its site of attachment to the user by
using an orientation of the IMU where the x axis is looking downwards,
using a number of posture changes from positive to negative values along the x axis of the acceleration to differentiate an IMU attached to the left or right wrist or lower arm from an IMU attached to the torso or left or right shank or ankle, and/or
using gyroscope total energy to differentiate an IMU attached to the torso from an IMU attached to the left or right shank or ankle.

14. The method according to claim 9, which does not require a step of configuring the IMUs before attaching the devices to the predefined body parts.
